SEQUENCE LISTING (2 sequences)

SEQ ID NO: 1 - DNA, 20 bp, Homo sapiens
agttcctcaaggcagggaag

SEQ ID NO: 2 - DNA, 20 bp, Homo sapiens
ctcagctgcactgtcttcag

(19) United States
(12) Patent Application Publication
(10) Pub. No.: US 2009/0215124 A1
(43) Pub. Date: Aug. 27, 2009

(54) Nucleic acid isolation methods and materials and devices thereof

(76) Inventors: Weidong Cao, Rockville, MD (US); Jerome P. Ferrance, Charlottesville, VA (US); James P. Landers, Charlottesville, VA (US)

Correspondence Address: BLANK ROME LLP, WATERGATE, 600 NEW HAMPSHIRE AVENUE, N.W., WASHINGTON, DC, 20037, US

(21) Appl. No.: 11/884,351

(22) PCT Filed: Feb. 15, 2006
(86) PCT No.: PCT/US06/05241

§ 371 Date: Jul. 24, 2008

Related U.S. Application Data
(60) Provisional application No. 60/653,203, filed on Feb. 15, 2005.

(51) Int. Cl. C12P 19/34; C07H 1/06; C08F 251/00; B01D 11/04

(52) U.S. Cl. 435/91.2; 536/25.4; 527/300; 422/255

(57) ABSTRACT

The present invention relates to methods for purifying nucleic acid from a sample using mild conditions that do not affect the chemical integrity of the nucleic acid. The method comprises contacting the sample with an matrix entrapped chitosan solid phase which is able to bind the nucleic acids at a first pH, and then extracting the nucleic acid from the solid phase by using an elution solvent at a second pH.

This application claims priority to U.S. Provisional Patent Application No. 60/653,203, filed Feb. 15, 2005, which is incorporated herein by reference in its entirety.

### FIELD OF THE INVENTION

The present invention relates to methods, compositions, and devices for isolating polynucleic acid from a sample. In particular, the present invention takes advantage of the ability of nucleic acid to reversibly bind chitosan to isolate the polynucleic acids from a sample.

### BACKGROUND OF THE INVENTION

There is a large demand for DNA analysis for a variety of purposes, which has lead to the desire for quick, safe, high throughput methods for the isolation and purification of DNA and other nucleic acids.

Samples used for DNA identification or analysis can be taken from a wide range of sources such as biological material such as animal and plant cells, faeces, tissue etc. Also, samples can be taken from soil, foodstuffs, water etc.

Existing methods for the extraction of DNA include the use of phenol/chloroform, salting out, the use of chaotropic salts and silica resins, the use of affinity resins, ion exchange chromatography and the use of magnetic beads. Methods are described in U.S. Pat. Nos. 5,057,426 and 4,923,978, EP 0512767 A1, EP 0515484B, WO 95/13368, WO 97/10331, WO 96/18731, and U.S. Pat. Publication No. 2001/0018513. These patents and patent applications disclose methods of adsorbing nucleic acids on to a solid support and then isolating the nucleic acids.

EP0707077A2 describes a synthetic water soluble polymer to precipitate nucleic acids at acid pH and release at alkaline pH. The re-dissolving of the nucleic acids is performed at extreme pH, temperature and/or high salt concentrations, where the nucleic acids, especially RNA, can become denatured, degraded or require further purification or adjustments before storage and analysis.

WO 96/09116 discloses mixed mode resins for recovering a target compound, especially a protein, from aqueous solution at high or low ionic strength, using changes in pH. The resins have a hydrophobic character at the binding pH and a hydrophilic and/or electrostatic character at the desorption pH.

Since the advent of micro total-analysis-systems (μ-TAS) (also known as “labs-on-a-chip” systems or microfluidic devices), microscale analytical chemistry has gained popularity for performing high throughput operations, including nucleic acid analysis, such as polymerase chain reaction (PCR), which creates great demands for a nucleic acid purification system that is capable of operating under mild conditions native to a biological system. A μ-TAS should have the capability to sequentially execute the numerous steps that almost always involve analysis of even the simplest biological or environmental samples. Invariably, this includes sample preparation steps prior to sample introduction, separation and detection. Use of a miniaturized device with sample in-answer out capabilities for sample analysis provides numerous advantages such as rapid analysis, low sample requirement, and automation, which are very conducive to biological analysis and, potentially, to point-of-care-testing applications.

Traditional genomic analysis exemplifies this notion because assays almost invariably involve purification of DNA from sample, target amplification by the polymerase chain reaction (PCR) or some analogous method, followed by electrophoretic size separation of the amplified fragments, hybridization, or direct fluorescence measurement. The implementation of these separate processes on microchips has been demonstrated to be an effective approach for DNA analysis. Electrophoretic separation of DNA on microchips has been demonstrated to provide high separation resolution in very short analysis times and is currently the gold standard. To achieve efficient PCR of DNA originating in biological matrices requires that the DNA be purified to remove all the PCR inhibitors—these exist in abundance in many biological samples, especially whole blood. Consequently, a fully-functional microdevice capable of PCR directly from samples then separation of the amplified products will require rapid, effective DNA extraction and purification.

DNA purification on microchips has been achieved through solid phase extraction (SPE) using silica absorption of DNA under chaotropic conditions. Christel et al. (Journal of Biomechainical Engineering 1999, 121:22-27) first reported DNA extraction on microchips by fabricating silicon dioxide pillars in the micro channel. Some of the present inventors have developed DNA purification on microchips using silica beads, sol-gel stabilized silica beads, and sol-gel only in micro-chambers to form the extraction column. Using silica-based SPE to extract DNA, biological samples are dissolved in a chaotropic solution, such as 6 M guanidine-HCl. Flow through the solid phase in the presence of the chaotrope enhances DNA interaction with the silica, primarily driven through hydrogen bonding and potentially some hydrophobic interaction. Proteins that have been adsorbed are eluted from the SPE column with an isopropanol solution, and the DNA eluted with low ionic strength buffer. For conventional purification of DNA from biological sources, this approach represents the most widely utilized and accepted method. It is rapid (with spin-based devices), the DNA extraction efficiency is acceptable for most applications, and the reagents that interfere with PCR (guanidine and ispopropanol) are not problematic because the method is stand-alone and carried out off-line. With μ-TAS, however, the desire to execute DNA extraction, PCR, and separation/detection sequentially entails that contamination of the PCR chamber with these reagents from the extraction process can be problematic.

Alternative approaches have been developed to avoid the use of chaotropic/organic reagents in the DNA purification process. Nakagawa et al. (J Biotechnol 2005, 116:105-111) used an aminosilane-modified open channel to extract DNA from whole blood on a microchip. Unlike silica-based SPE, this method exploited the fact that the amino group is cationic below its pKₐ (in the pH 9.5 range) and neutral above its pKₐ. This provided a means of creating a DNA capture state and DNA release state on the surface mediated by simple changes in pH. Extraction of DNA was achieved at pH 6.0 via electrostatic interactions with the charged phosphate backbone of the DNA. Proteins that bound to the cationic surface were washed from the channel with aqueous buffer, and the DNA released by increasing the pH to 10.6. The attractive aspect of this method is the ability to completely avoid the use of reagents that act as PCR inhibitors (i.e., isopropanol or chaotropic salts). However, problematic to subsequent PCR is the high pH (10.6) that is required for neutralizing the aminosilane surface and releasing the DNA—this is incompatible with the PCR process and certainly limits the PCR-readiness of the eluted DNA. In addition, to capture DNA in the covalently-modified open channel, extensive channel length (10.4 cm) was required with 100 μm deep and 300 μm wide channels. Subsequently, DNA was eluted in a volume of 45 μl, on the order of 100-fold larger than would be used in a μ-TAS, where PCR of solutions in the nanoliter range is sought.

Likewise, U.S. Pat. No. 6,914,137 discloses a method for extracting nucleic acids from a biological material using “charge switching materials.” In this work, the more moderate pKₐ associated with the protonatable nitrogen of the imidazole group (pKₐ=6.7) provided a matrix that was more amenable to DNA extraction. The surface charge could be altered from a DNA capture state at a pH of ˜6 to the DNA release state at pH 8.5, where purified nucleic acids eluted instantly into a low salt buffer. While this protocol was advantageous because it was exclusively aqueous, the existence of carboxyl groups in histidine make the system more susceptible to protein absorption. Moreover, the specific interaction of some proteins with histidine through the imidazole functional group has been reported. These factors could compromise the efficiency of the DNA purification process. Further, this patent also discloses chitosan as a charge switching material; however, chitosan, by itself, binds nucleic acid too strongly resulting in low yield upon elution at an alkaline pH.

Therefore, there remain a need for processes, compositions, and devices for purifying nucleic acids with high efficiency, using mild condition and chemicals, and capable being used in a μ-TAS.

### SUMMARY OF THE INVENTION

The present invention provides methods for the extraction of nucleic acid from a sample. The method comprises contacting the sample with a solid phase which is able to bind the nucleic acids at a first pH with minimal protein binding, and releasing the nucleic acid from the solid phase by using an elution solvent at a second pH.

The solid phase material is chitosan immobilized to a matrix, which has an overall positive charge. It may be possible (though not preferred), however, that the solid phase as a whole could be negatively charged or neutral in charge, but have areas of predominantly positive charge to which the nucleic acid can bind.

The matrix-immobilized chitosan is preferably a chitosan/sol-gel that may be formed from crosslinking chitosan with 3-glycidyloxypropyl trimethoxysilane (GPTMS). The matrix-immobilized chitosan is preferably coated on a bead, such as a silica or magnetic bead.

In an embodiment, the matrix-immobilized chitosan is used to purify nucleic acid using a μ-TAS device. In this embodiment, the matrix-immobilized chitosan may be attached directly to the wall of a microchamber or microchannel. Alternatively, the microchamber or microchannel contains matrix-immobilized chitosan coated beads through which a sample passes.

The nucleic acid purified by the method of the present invention may be used in further processing, reactions, or analysis, which may occur in the same container or reservoir. One main advantage of the matrix-immobilized chitosan resides in its low affinity for proteins; thus, further processing of the nucleic acid requiring proteinaceous reactants (such as enzymes) does not require the removal of the solid phase. In an embodiment, the matrix-immobilized chitosan is used to capture nucleic acid for polymerase chain reaction (PCR) or other analysis nucleic analysis steps, such as hybridization or other reactions. In this embodiment, the captured nucleic acid may or may not be released from the matrix-immobilized chitosan prior to the vitiation of the PCR.

In one preferred embodiment, the captured nucleic acid is released from the matrix-immobilized chitosan prior to PCR but not mobilized from the bead bed area. Here, PCR takes place with the captured nucleic acid desorbed from but still in the presence of the solid phase.

In another preferred embodiment, the captured nucleic acid is not released from the from the matrix-immobilized chitosan prior to PCR. Here, PCR takes place with the captured nucleic acid attached to the solid phase.

The advantage of these preferred embodiments resides in the fact that nucleic acid purification and amplification takes place in the same reservoir, be it in a test tube, microfuge tube, or a microfluidic chamber; saving the steps of involved in mobilizing the nucleic acid from the solid phase area or separating the nucleic acid from the solid phase, thereby minimizing the amount of nucleic acid losses through processing.

### BRIEF DESCRIPTION OF THE DRAWINGS

The foregoing background and summary, as well as the following detailed description of the preferred embodiment, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a drawing of the use of magnetic beads coated with matrix-immobilized chitosan in a μ-TAS.

FIG. 2 is a drawing showing (A) the high density open channel microchip with a binary lamination design; blue ink was used to aid visualization; and (B) Side view illustration of microchip and manual pressure device for flow generation.

FIG. 3 is a graph showing the pH dependence of DNA elution from the matrix-immobilized chitosan coated on silica beads.

FIG. 4 is a graph showing DNA and protein profiles during extraction of human genomic DNA from serum by matrix-immobilized chitosan coated silica beads.

FIG. 5 is a graph showing DNA extraction capacity of matrix-immobilized chitosan coated beads.

FIG. 6A is a graph showing DNA extraction profiles for λ-phage DNA (gray) and human genomic DNA (black) using the matrix-immobilized chitosan coated on the walls of the open channel binary lamination design microchip.

FIG. 6B is a graph showing reproducibility of human genomic DNA extractions in four different microchips.

FIG. 7 is a graph showing the average DNA breakthrough from continuous loading of human blood on three separate microchips—this breakthrough curve was used to determine the DNA capacity for the chitosan coated microchips.

FIG. 8 is a graph showing electropherogram traces of PCR products after amplification of the gelsolin gene from human genomic DNA template. Trace A shows separation of a DNA marker; trace B shows amplification from a positive control using purified human genomic DNA; trace C and D show amplification of DNA from blood extracted on chitosan coated microchips; trace E shows a negative control with no template DNA.

FIG. 9 illustrates the lack of inhibitory effects of the matrix-immobilized chitosan coated magnetic beads on real-time PCR.

FIG. 10A shows the linear relationship between template starting copies and threshold cycle with chitosan coated magnetic beads included in the reaction. No difference was seen between this curve and a control curve generated without added beads.

FIG. 10B shows the real-time curves for amplifications of standard amounts of DNA template.

FIG. 10 is a graph showing successful extraction of DNA using matrix-immobilized chitosan coated magnetic beads. Bar 1 is the DNA recovered from the load solution after loading; bar 2 is the DNA recovered in the wash solution; Bar 3 is the DNA recovered during elution.

FIG. 11 shows electropherograms of products from IR mediated microchip PCR amplifications of a 500 bp product of lambda phage DNA. Graph A shows amplification of DNA captured then released from chitosan coated magnetic beads placed in the PCR chamber on the microchip as shown in FIG. 1. Graph B shows a positive control with lambda phage DNA; Graph C shows two negative control amplifications.

FIG. 12 shows electropherograms of products from IR mediated microchip PCR amplifications of a 64 bp product from the TPOX gene of human genomic DNA. Graph A shows amplification of DNA captured then released from matrix-immobilized chitosan coated magnetic beads placed in the PCR chamber on the microchip as shown in FIG. 1. Graph B shows a positive control with human genomic DNA; Graph C shows a negative control amplification. a

FIG. 13 an electropherogram of products from an IR mediated microchip PCR amplification mixed with a DNA standard for amplified fragment size determination. Lysed human blood was loaded into the microchip, and the DNA captured on the matrix-immobilized chitosan magnetic beads placed in the PCR chamber on the microchip as shown in FIG. 1. Contaminating substances were washed away then the DNA released in PCR buffer and directly amplified in the PCR chamber to produce the expected 64 bp product.

FIG. 14 is a drawing showing the process of entrapping the chitosan within a sol gel type matrix.

### DETAILED DESCRIPTION OF THE PREFERRED EMBODIMENT

The present invention relates to methods for purifying nucleic acid from a sample using mild conditions that do not affect, even temporarily, the chemical integrity of the nucleic acid. The method comprises contacting the sample with a solid phase which is able to bind the nucleic acids at a first pH, and extracting the nucleic acid from the solid phase by using an elution solvent at a second pH. In the binding step, the solid phase selectively binds the nucleic acid and retained it thereon. The binding pH is preferably about 3-6, more preferably about 4-5, and most preferably about 5. The elution pH is preferably greater than about 8, more preferably about 8-10, and most preferably about 9. Preferably, the elution step is carried out in the substantial absence of NaOH, preferably also the substantial absence of other alkali metal hydroxides, more preferably the substantial absence of strong mineral bases. Substantial absence means that the concentration is less than 25 mM, preferably less than 20 mM, more preferably less than 15 mM or 10 mM.

Preferably the temperature at which the elution step performed is no greater than about 70° C., more preferably no greater than about 65° C., 60° C., 55° C., 50° C., 45° C. or 40° C. Most preferably, the same temperatures apply to the entire process for both the adsorption and the elution step. The elution step, or the entire process, may even be performed at lower temperatures, such as 35° C., 30° C., or 25° C. Most preferably, the entire process occurs at room temperature.

Furthermore, the elution step preferably occurs under conditions of low ionic strength, suitably less than about 1M or 500 mM, preferably less than about 400 mM, 300 mM, 200 mM, 100 mM, 75 mM, 50 mM, 40 mM, 30 mM, 25 mM, 20 mM, or 15 mM, most preferable less than about 10 mM. The ionic strength may be at least about 5 mM, more preferably at least about 10 mM. These ionic strengths are also preferred for the binding step.

The use of such mild conditions for the elution of nucleic acid is especially useful for extracting small quantities of nucleic acid, as the extracted DNA or RNA can be transferred directly to a reaction or storage tube without further treatment steps. Therefore loss of nucleic acid through changing the container, imperfect recovery during further treatments, degradation, denaturation, or dilution of small amounts of nucleic acid can be avoided. This is particularly advantageous when a nucleic acid of interest is present in a sample (or is expected to be present) at a low copy number, such as in certain detection and/or amplification methods.

The preferred solid phase contains chitosan which is the product of alkaline hydrolysis of abundant chitin produced mainly in the crab shelling industry. Chitosan, a biopolymer, is soluble in dilute (0.1 to 10%) solutions of carboxylic acids, such as acetic acid, is readily regenerated from solution by neutralization with alkali. In this manner, chitosan has been regenerated and reshaped in the form of films, fibers, and hydrogel beads. In the present invention, chitosan is preferably immobilized to a matrix, preferably of another polymer, more preferably of a sol-gel. Immobilized, as used herein, means that the chitosan may be physically contained in the matrix or may be chemically linked to the matrix material. Physical containment of the chitosan means that the chitosan is physically trapped within the matrix without being chemically bonded to the matrix material. On the other hand, the chitosan may also be chemically bonded to the matrix material through ionic, covalent, or other chemical bonds.

In one embodiment of the present invention, the chitosan forms a copolymer with another polymer, thereby being entrapped in a matrix. The copolymerization may contain various crosslinking to form a solid or a gel.

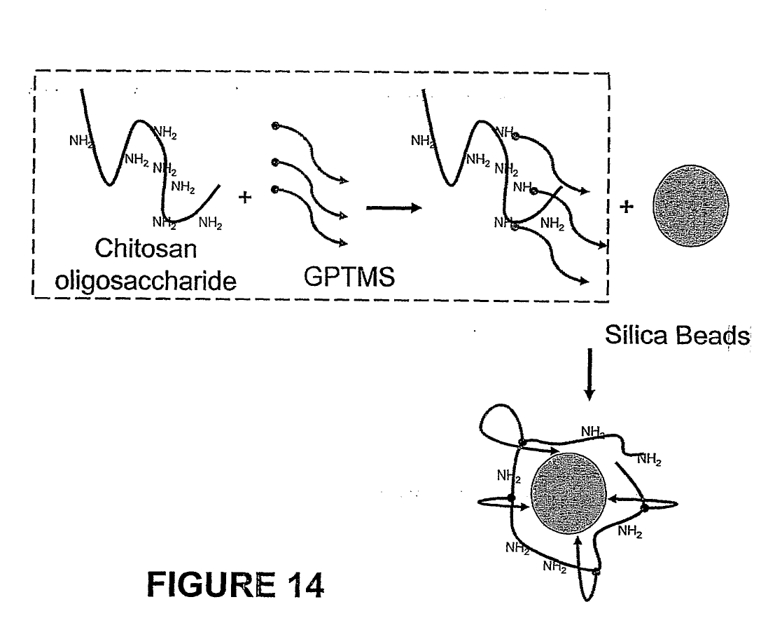

In a preferred embodiment, the chitosan and the matrix material are copolymerized to form a copolymer, preferably a chitosan/sol-gel composition. In this embodiment, the sol-gel are preferably formed from silanes, such as aldehyde triethoxysilanes, aminopropyl triethoxysilanes, 3-glycidyloxypropyl trimethoxysilane (GPTMS), most preferably GPTMS. The sol-gel is formed either under the acidic condition pH from 0.1 to 6), most preferably between 2-5, or under the basic condition pH from 8 to 12, most preferably between 8 to 10. The addition of 0.1% to 50% methanol or ethanol is preferably accelerates the form the chitosan/sol-gel copolymer. The reaction temperature is from 10 centigrade to 90 centigrade, preferably at 30 centigrade. The reaction time of forming of chitosan/sol-gel copolymer is from 1 min to 64 hours, depending on the pH value, reaction temperature, and concentration of methanol and ethanol. For example, a chitosan/sol gel composition may be made as shown in FIG. 14. Here, chitosan and GPTMS are polymerized to form a cross-link copolymer (chitosan/sol gel). The copolymer may be used alone or coated onto a bead.

The matrix-immobilized chitosan may be immobilized onto solid supports (e.g. beads, particles, tubes, wells, probes, dipsticks, pipette tips, slides, fibers, membranes, papers, celluloses, agaroses, glass or plastics) via adsorption, ionic or covalent attachment. For example, a chitosan/sol-gel material may be immobilized on to and coats silica beads for use in nucleic acid purification as shown in FIG. 14.

The solid support, especially beads and particles, may be magnetizable, magnetic or paramagnetic. This can aid removal of the solid phase from a solution containing the nucleic acid, prior to further processing or storage of the nucleic acid, or aid in the control of the magnetic particles via a magnetic field as discussed below.

In a preferred embodiment, the matrix-immobilized chitosan composition is used to purify nucleic acid in a μ-TAS. There are many formats, materials, and size scales for constructing μ-TAS. Common μ-TAS devices are disclosed in U.S. Pat. Nos. 6,692,700 to Handique et al.; 6,919,046 to O'Connor et al.; 6,551,841 to Wilding et al.; 6,630,353 to Parce et al.; 6,620,625 to Wolk et al.; and 6,517,234 to Kopf-Sill et al.; the disclosures of which are incorporated herein by reference. Typically, a μ-TAS device is made up of two or more substrates that are bonded together. Microscale components for processing fluids are disposed on a surface of one or more of the substrates. These microscale components include, but are not limited to, reaction chambers, electrophoresis modules, microchannels, fluid reservoirs, detectors, valves, or mixers. When the substrates are bonded together, the microscale components are enclosed and sandwiched between the substrates.

The matrix-immobilized chitosan is contained within a microscaled component of the μ-TAS. This may be accomplished by having beads or other support material coated with the matrix-immobilized chitosan inside the microscaled component, or immobilizing the matrix-immobilized chitosan directly on to the wall of the microscaled component. Either way, the microscaled component may be used to capture nucleic acid in a sample that passes into or through the microscaled component.

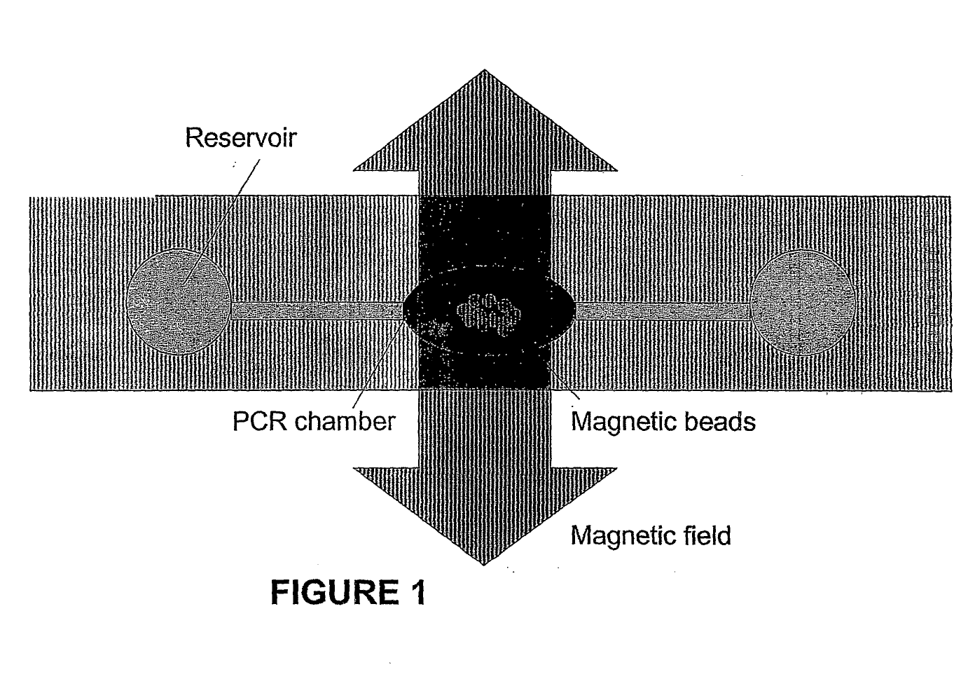

In a preferred embodiment, magnetic beads coated with matrix-immobilized chitosan is used in a PCR chamber as shown in FIG. 1, where DNA capture (binding), elution, and PCR all takes place in the same chamber. In this configuration, a magnet is used to control and move the beads during the capture, wash, and elution steps. For example, during those steps, a magnetic field may be used to “stir” the beads within the chamber. After elution, the purified nucleic acid may be amplified by PCR in the same chamber. During PCR, the magnet immobilizes the beads in against the wall to remove them from the microarea where thermocycling occurs.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### Example 1

### Purification of DNA Using Silica Beads Coated with Chitosan/Sol Gel Copolymer

Before coating, silica beads were cleaned in piranha solution (2:1, H₂SO₄:H₂O₂) at 70° C. for 10 min. Then the beads were washed to neutrality with water and dried thoroughly. Chitosan coating of the treated silica beads was accomplished through incubation with 0.1% GPTMS, which provides the crosslinker between the silica beads and chitosan, and 1% chitosan. The beads were then cleaned with 10 mM acetic acid and water to wash unbound chitosan off the beads.

The DNA extraction procedure consisted of load, wash, and elution steps. In the load step, 60 μg of chitosan-coated silica beads were mixed with a solution containing DNA and allowed to react for 10 min in a polypropylene tube. After centrifugation at 5000 rpm for 10 sec, the load solution was removed from the tube. The beads were further washed by 20 μl of 10 mM MES (pH 5.0) buffer for 5 min. The washing solution was removed after another brief centrifugation. Then 10 μl solution buffer (10 mM Tris-buffer at pH 9.0, 50 mM KCl) was added to the tube. Following a 5 min. incubation, the elution buffer containing the eluted DNA was removed from the tube after centrifugation. For extractions from serum solutions, 5 μL of serum was mixed into 20 μL of load buffer containing the DNA before addition of the coated beads.

Extracted DNA solutions from blood samples were directly mixed with PCR solution and amplified using a Perkin-Elmer Thermocycler (Santa Clara, Calif.) and standard PCR protocols. This involved a pre-incubation step at 95° C. for 3 min, up to 35 cycles with 94° C. for 30 sec/64° C. for 30 sec/72° C. for 30 sec followed by final extension at 72° C. for 3 min. A 139-bp fragment of the human genomic gelsolin gene was amplified with primers 5′-AGTTCCTCAAGGCAGGGAAG-3′ (SEQ ID NO: 1) and 5′-CTCAGCTGCACTGTCTTCAG-3′ (SEQ ID NO: 2) purchased from MWG BioTech (High Point, N.C.). All amplified samples were separated and analyzed on a Bio-Analyzer 2100 (Agilent Technologies, Palo Alto, Calif.) using DNA 1000 kits.

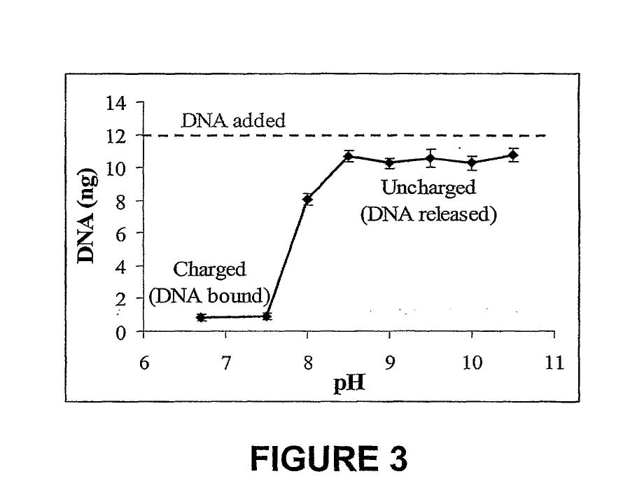

Initial testing of the chitosan-coated silica beads as a DNA extraction phase involved microcentrifuge tube-based extractions of lambda bacteriophage DNA (λ-DNA) whose entire genome is 48 kbp in length. λ-phage DNA (12 ng) was added to a slurry of chitosan-coated beads in the presence of 10 mM Tris buffer containing 50 mM KCl at pH 5.0. After incubation and centrifugation, to pellet the beads, the supernatant was removed and the DNA remaining in solution was measured using a fluorescence assay. Less than 1 ng of DNA remained in solution indicating that greater than 90% of the DNA had been extracted from solution by the chitSP beads (data not shown). Capture of the DNA was followed by release of the DNA from the beads using different pH values for the elution buffer; the data from these experiments (n=5 at each pH) are shown in FIG. 3. With elution buffer pH values of 6.7 and 7.5 (values that lie near the pKₐ of chitosan) release of the DNA from the beads after 5 min was poor (0.82±0.2 ng and 0.91±0.2 ng, respectively). These results are in agreement with those from Bozkir et al. (Drug Deliv. 2004, 11:107-112) who observed that, at pH 7.5, DNA was released from chitosan at a very slow rate over the course of 24 hours, presumably the result of elution at a pH too close to the pKₐ (at a pH of 7.5, roughly 10% of the amino groups are still protonated). Although Bozkir et al. also point out that the kinetics of DNA release is dependent on the degree of chitin deacetylation. At pH 8.0, however, a sharp increase is observed in the mass of DNA released in only 5 min, with 8.04±0.33 ng of DNA eluted (n=5). Increasing the elution buffer pH beyond 8.0 showed a plateau effect with an average of 10.5 ng of DNA eluted in higher pH buffer, corresponding to a DNA extraction efficiency of 87.5±2%. Since it was clear that no selective advantage was gained by eluting at higher pH values, a pH of 9.0 was chosen for elution for further experimental works. Thus, unlike the high pH required with the amino-silane modified surface (Nakagawa et al., J. Biotechnol. 2005, 116:105-111), chitosan-coated surfaces allow for DNA release at a pH closer to that required for PCR.

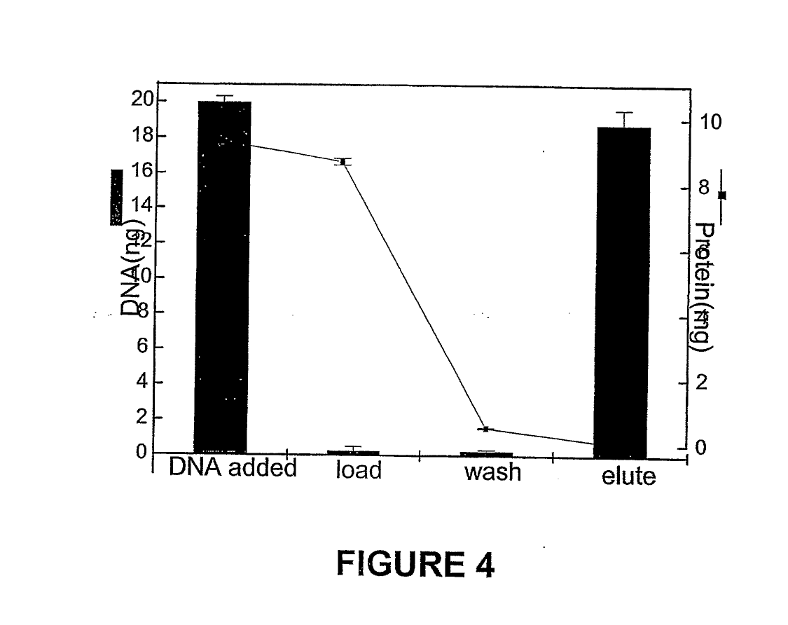

With the optimal elution pH values determined, the chitosan-coated silica beads were used to extract human genomic-DNA from a mixture containing DNA (20 ng) and serum (5 μL); this mixture allowed us to investigate the effect of a heterogeneous protein mixture on DNA extraction. The graph in FIG. 4 shows the DNA profile obtained from four extractions performed using 120 μg of chitSP beads. Upon removal of the supernatant by centrifugation after a 10-min incubation, less than 0.5 ng of human genomic-DNA remained in the load solution as measured by a fluorescence assay. A wash step was used to remove any protein or unbound DNA associated with the beads or tube—the fact that no detectable DNA was recovered from the beads in the wash step corroborated the strength of the interaction between DNA and chitosan. The DNA bound to the chitosan beads was eluted using Tris buffer at pH 9.1, a pH purposefully chosen to meet the needs of subsequent PCR and yielded an extraction efficiency of 92.1±4.0% (n=4).

FIG. 4 also contains a protein elution profile for this extraction method to demonstrate the low protein binding character of the chitSP beads. To provide sufficient protein for quantitation, 0.1 mL of serum was mixed in 1 ml of load buffer then 5 mg of chitosan beads were added and the extraction procedure performed as normal. The distribution of protein in the load, wash and elution solutions was quantified using the BCA™ protein Assay Kit. Before extraction, about 9 mg of protein was measured in the serum solution sample. Greater than 90% of the protein (n=3) remained in the load solution with most of the remainder being removed in the wash step. The amount of the protein in the elution buffer was as small as 19 μg, which is less than 3% of the amount of protein absorbed on the same amount of uncoated silica beads. These results confirmed the low protein adsorption ability of chitosan, indicating that the chitosan coated beads could successfully purify DNA into an essentially protein-free state, ready for further processing and analysis.

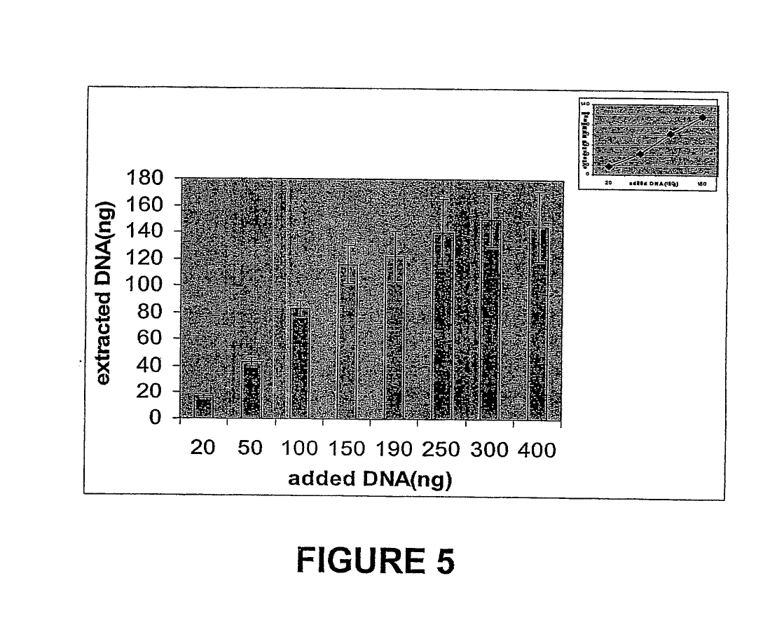

To determine the DNA capacity of the chitSP beads, the amount of DNA needed to saturate the binding sites associated with 60 μg of beads was determined. Solutions containing λ-DNA, ranging from 20-400 ng in the same volume of load buffer, were extracted and the amount of DNA remaining in the load solutions was determined using the fluorescence assay. The results as provided in FIG. 5 show that extraction is linear in the 1-150 ng range (see inset), beyond which the binding capacity plateaus. This indicates that the chitSP beads have a capacity of 2.4 mg DNA/g chitSP beads, similar to the 4 mg DNA/g particles capacity of the commercially available MagneSil particles.

### Example 2

### DNA Purification in Multi-Channel Microchips Coated with Chitosan/Sol Gel Copolymer

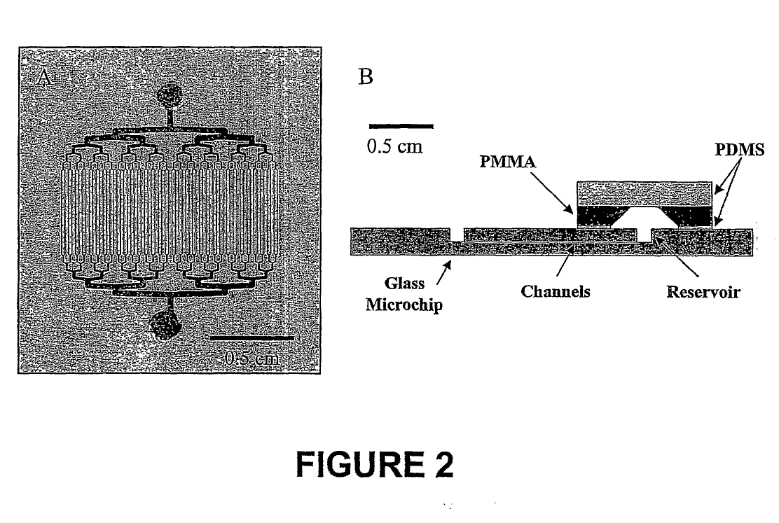

The multi-channel extraction microchips were fabricated using standard photolithographic techniques. From the sample inlet, channels were divided through binary lamination according to the method of He et al. (Anal. Chen. 1998, 70:3790-3797) until 64 parallel channels were obtained, then rejoined into one channel at the outlet reservoir as shown in FIG. 2A. A 1.1 mm diameter access hole was drilled at each reservoir. A complete device was formed by thermal bonding of the etched plate with a cover plate at 640° C. To ensure that sample solution evenly diffused from a single inlet channel into multi channels, the inlet and outlet architecture was designed similar to that of He et al. With splitting of the channel, the channel dimensions decreased as the ratio of 2ⁿ. This design provided the same linear flow velocity at all points. The final number of channels (C) serving for DNA extraction was expressed by C=2ⁿ. In this experiment, we designed 64 channels for DNA extraction with each channel 0.5 cm long by 17 μm depth, with a top width of 83 μm, and a width of 33 μm at the bottom. These dimensions resulted in a surface area-to-volume ratio (SAN) of 151 mm⁻¹ and a combined flow resistance to viscosity ratio of 1.1×10⁻⁵ μm⁻³. Before coating, the channels were cleaned by piranha solution at 70° C. for 10 min. The coating process was identical to that used for silica beads, using the channel filled with solution for the incubation with 0.1% GPTMS, to act as the crosslinker to the channel wall, and 1% chitosan before rinsing with 10 mM acetic acid and water to remove unbound chitosan. Mineral oil was added to the reservoirs to prevent evaporation of the solution in the channels during the coating process.

The use of the 64 parallel open channels generated significantly less back pressure for flow of solutions through the microchip compared to a bead-packed extraction column. To pass solution through the chip, the simple, manual pressure-driven device shown in FIG. 2B was designed and fabricated. A 5-mm diameter hole was drilled at the bottom of a 5 mm thick poly(methylmethacrylate) (PMMA) plate, with 1- to 5-mm variable diameter holes drilled into the top. A 0.25 mm thick PDMS film with a 5 mm diameter hole was adhered to the bottom of the PMMA plate. Another 2-mm thick PDMS layer was adhered to the top of the PMMA plate. The device was placed on the inlet reservoir, and solution was flowed through microchip by pressing on the top PDMS layer. The flow rate was adjusted by varying the diameter of the top hole in the PMMA sheet. The solution was collected at the outlet reservoir. This device allowed manual pressure-driven flow control of solutions in the microchip, and its ease of use was accentuated by the low flow resistance of the microchip.

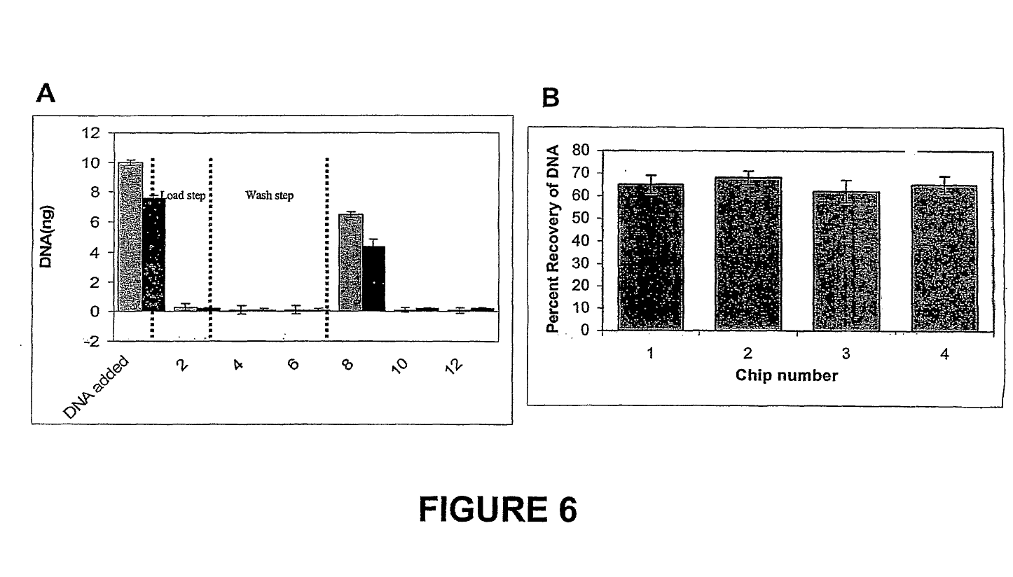

FIG. 6A shows the extraction profile of λ-DNA (gray bars) and human genomic DNA (black bars) on the binary laminated design microchip. All solutions were injected into the channels using the manual pressure device shown in FIG. 1 at a flow rate of about 1 μL/min. As shown in FIG. 6A, only negligible amounts of either type of DNA were detectable in the load or wash buffers. DNA was eluted with 6 μL of elution buffer (10 mM Tris+50 mM KCl at pH 9.0) with 2 μL aliquots collected for quantitation by the fluorescence assay. Interestingly, 65±5% of the loaded λ-DNA was detected in the first 2 μL fraction of elution buffer, and no obvious DNA was detected in the subsequent elution fractions. Further investigations showed that about 10% of λ-DNA was in the first 1.0 μL fraction of elution buffer and about 60% λ-DNA was in the second 1.0 μL fraction (data not shown). However, with only 72% total recovery, some of the λ-DNA was apparently retained in the channel and was not removed by the pH-induced release method. The retention mechanism was not investigated further. For pre-purified human genomic DNA, the extraction profile was similar to that of the λ-DNA profile. The extraction efficiency was 68±9%, with 63±9% of loaded human genomic DNA eluted in the first 2 μL fraction. These results indicated that using the high-density pattern provided sufficient SA/V to capture DNA and that the chitosan charge-switching allowed quick release from the SPE surface with a simple pH change.

Further studies showed reproducible extraction efficiencies between chips (FIG. 6B), using 6.7 ng of human genomic DNA as the loaded sample and evaluating the performance with three extractions per chip with four chips. The average extraction efficiency for the four chips was 65±5%, with the excellent reproducibility not surprising as a result of the precise and reproducible way that the surface area for extraction was defined by the channel pattern and fabrication process.

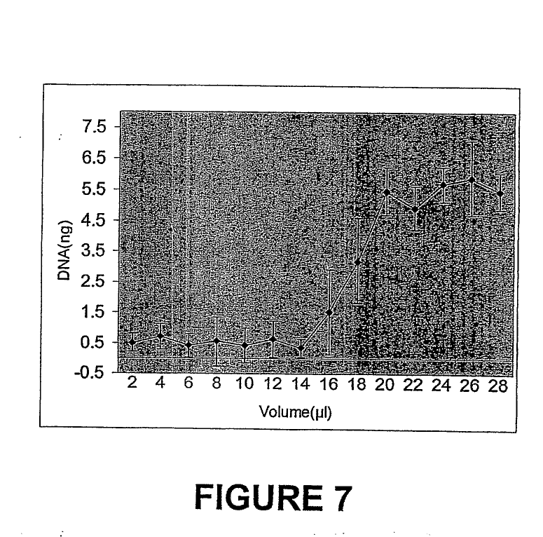

To evaluate the capacity of these microchips for DNA extractions from blood, 5 uL of human blood from a healthy volunteer was lysed in 45 μL of 50 mM MES buffer containing 1% triton X-100 and 2 mg/ml proteinase K for 30 min at room temperature. The DNA concentration in this load sample was determined to be 3.2 ng/μL (n=4) for a total mass of DNA of 160 ng. The lysed blood sample was loaded into the chip and the load solution fractions were collected in 2 μL aliquots at the microchip outlet for fluorescence analysis. FIG. 7 shows the DNA concentration in each of the fractions collected at the outlet. As seen from the plot, almost all of the DNA was captured from the first 14 μL of sample loaded. After that, the amount of DNA remaining in the collected fractions gradually increased until it reached a plateau value. Using a breakthrough curve analysis, the DNA extraction capacity of the microchip from whole blood was determined to be 48.7 ng. Using the same breakthrough method, the DNA extraction capacity for purified human genomic DNA has measured about 58 ng for the microchip. The comparable extraction capacity confirms that excessive amounts of protein in whole blood do not significantly compromise the DNA capture ability of the chitosan phase as indicated by the previous results for extraction of DNA from serum solutions.

### Example 3

### Microchip-Based Purification of Genomic DNA from Blood Samples

The extraction efficiency of the chitosan-coated open channel microchip was determined above for prepurified DNA, but while the proteins in whole blood did not significantly affect the capacity of the microchip, the extraction efficiency from whole blood had to be determined. A 4 μL whole blood sample was mixed with 36 μL of lysis buffer, then 2 μL of this mixture (0.2 μL of the original blood sample) was loaded onto the microchip at the rate of ˜1 μL/min. Following the usual wash step, the DNA was released by elution with 2 μL of elution buffer which was determined to contain 5.1±0.3 ng (n=3). Assuming that 5000 white cells were present per μL of blood sample, the amount DNA in the 0.2 μL of loaded blood was estimated to be 7.0 ng. The whole blood DNA extraction efficiency by microchip, therefore, was 75±4% (n=3). This demonstrated that the chitosan-coated open channel microchip design could be used to successfully extract DNA from a complicated biological sample with high extraction efficiency.

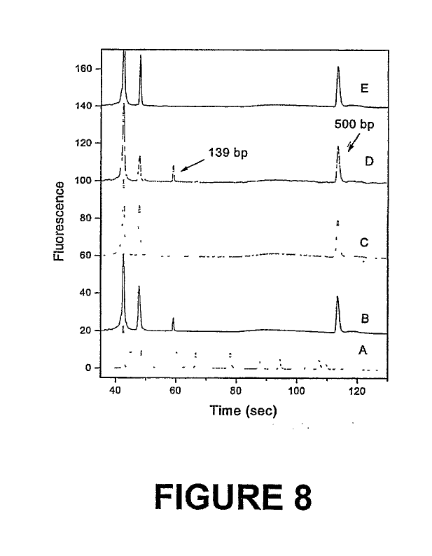

Finally, to determine if the extracted DNA from whole blood was PCR-ready, the elution buffer was directly added to a PCR reaction mixture and a 139-bp fragment from the gelsolin gene was amplified via conventional PCR. Gelsolin is an important protein in the “gel” to “sol” transformation in cell motility, functioning to sever and cap actin filaments in a way that regulate the length of filaments involved in cell structure, motility, apoptosis, and cancer. The DNA extracted from whole blood on the microchip was amplified and the products were subsequently separated using microchip electrophoresis. FIG. 8 shows the electropherograms of the PCR products amplified from the human genomic DNA. Trace A in FIG. 8 shows the DNA sizing standard and trace B shows the electrophoretic profile of the positive control, consisting of 3.8 ng of purified human genomic DNA added as template in the PCR amplification. The amount of DNA added to the positive control was expected to be at the same level as that extracted from the whole blood. Traces C and D show the electrophoretic profiles of PCR products using template DNA purified from 200 nL of whole blood by the chitosan-coated microchannels. The peak heights of the gelsolin gene amplicon were comparable to the positive control. This indicates that the microchip-extracted DNA sample was pure enough for PCR amplification, despite the high complexity of the initial sample. Trace E in FIG. 8 shows the electrophoretic profile of the negative control using a DNA-free load buffer passed through the microchip.

### Example 4

### Purification and PCR of DNA with Chitosan/Sol Gel Coated Magnetic Beads in a Microchip

A microchip as disclosed above for FIG. 1 was constructed having chitosan/sol gel coated magnetic silica beads in a PCR chamber. The volume of PCR chamber was about 1.0 ul. Both the extraction and amplification were performed in the chamber. A permanent magnet was placed above the ellipse and used to control the beads during the load, wash, and elute steps. During PCR, the magnet resided at the top of the air pocket to hold the beads in place during thermocycling. The magnetic beads were kept in a mobile state in the PCR chamber (e.g., through a back and forth action) for 1 min. by changing the direction of the magnetic field during the bad, wash and elute steps. After each step, the beads were held on the wall of the CR chamber by the permanent magnet. The DNA was eluted using a PCR master mix (10 mM Tris, 50 mM KCl pH 9, 25 mM MgCl2, 0.2 μM each primer, 0.2 mM NTP and 0.1 U/μL Taq polymerase), and then thermocycled using the non-contact thermocycling system. PCR was carried out for 35 cycles in 12 min. Capillary electrophoresis was performed on the PCR product.

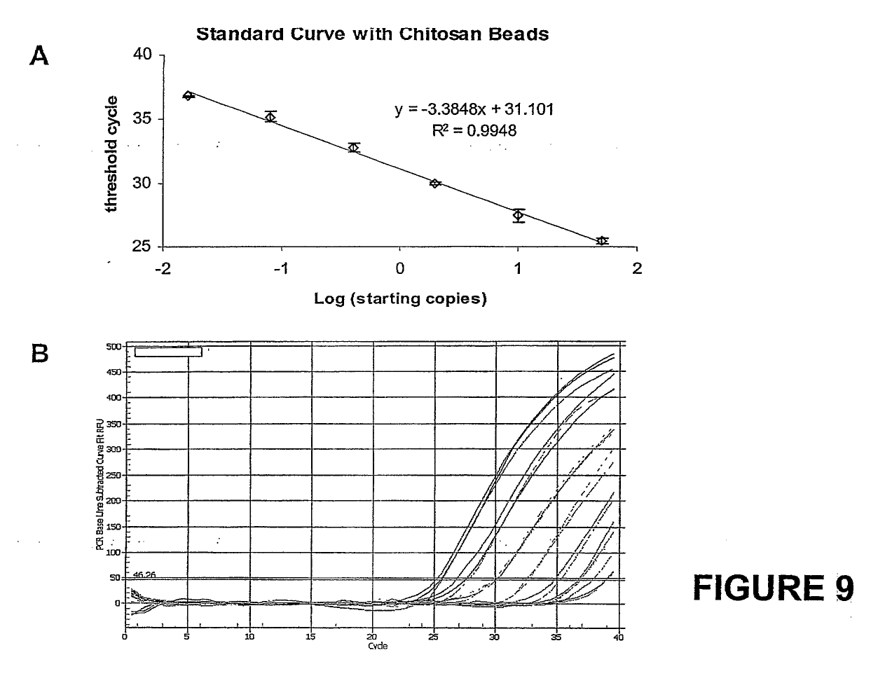

qPCR was performed using a VIC labeled Taqman probe to amplify a fragment from the human specific TPOX gene. 1 uL of 5 mg/mL beads were included in each 25 uL reaction. The data in FIG. 9 above shows (A) a standard curve starting with 50, 10, 2, 0.4, 0.08, 0.016 ng DNA along with (B) the real time fluorescence increase during the amplification. This data shows that the PCR is not inhibited by inclusion of the chitosan magnetic beads.

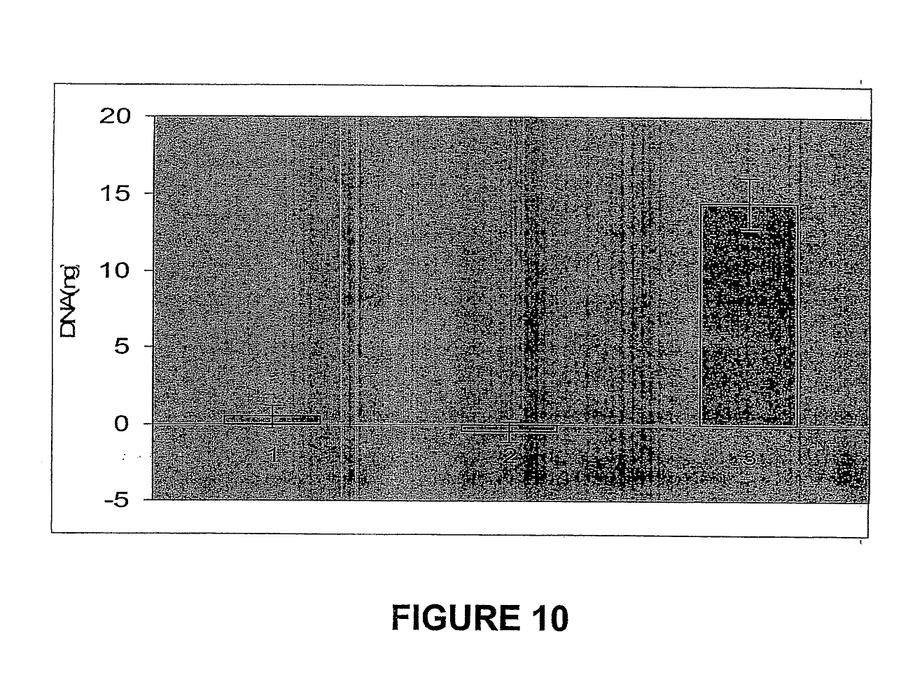

FIG. 10 shows the DNA recovery from an extraction with magnetic chitosan beads in a test tube to determine the purification efficiency of the magnetic beads. The extraction was performed in a tube with 2 uL of 30 mg/mL amount of beads and 20 ng of prepurified human genomic DNA was loaded onto the chitosan beads in 10 mM MES buffer, pH 5. The load, wash and elution solutions were collected and the amount of DNA present in each fraction was quantified using a fluorescence-based assay (Picogreen). These beads were determined to have an extraction efficiency of 77±11% (n=5).

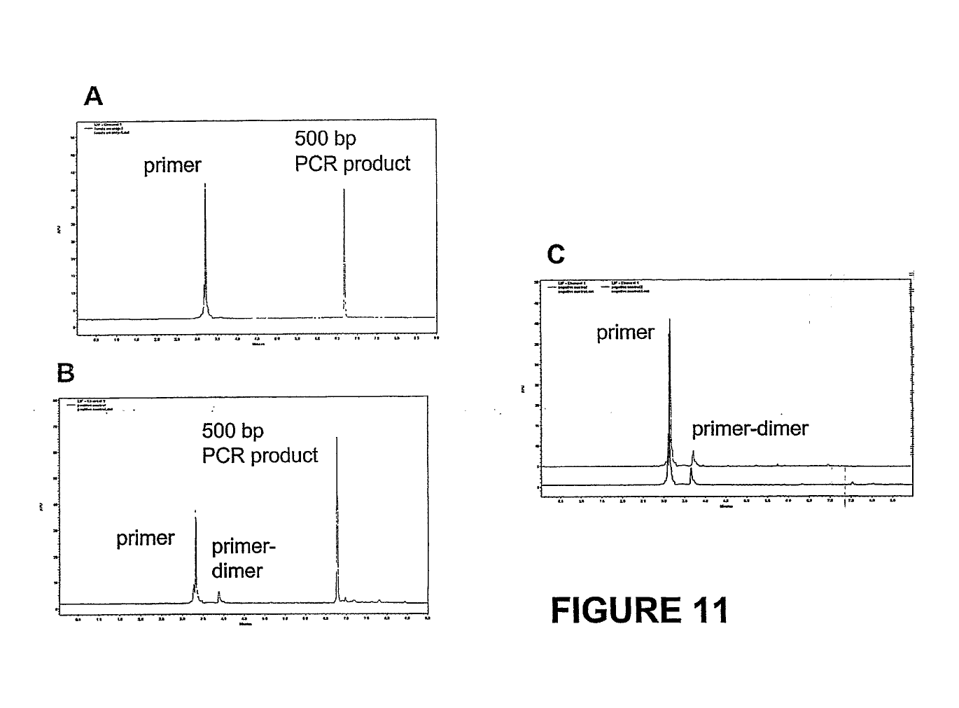

SPE-PCR was also performed in the same PCR chamber for Lambda SPE-PCR. FIG. 11 shows the result of the PCR reactions. In FIG. 11A, 5 ng of prepurified lambda DNA was loaded onto the magnetic chitosan beads in the PCR chamber. The DNA was eluted using PCR master mix (10 mM tris 50 mM KCl pH 9, 25 mM MgCl2, 0.2 μM each primer, 0.2 mM dNTP and 0.1 U/μL Taq polymerase) and then thermocycled using the non-contact thermocycling system. Capillary electrophoresis was performed on the PCR product and the separation shows the specific 500-bp fragment expected (FIG. 11A).

In FIG. 11B, 1 ng prepurified lambda DNA was added to the PCR master mix, same as stated above, along with 2 uL of 30 mg/mL amount of chitosan beads and PCR was performed using the non-contact thermocycling system. The presence of a 500-bp fragment indicates that the PCR was successful in the presence of the chitosan beads.

FIG. 11C shows the PCR master mix, without DNA. The beads were flowed into the chamber and non-contact PCR was performed, resulting in no specific amplification.

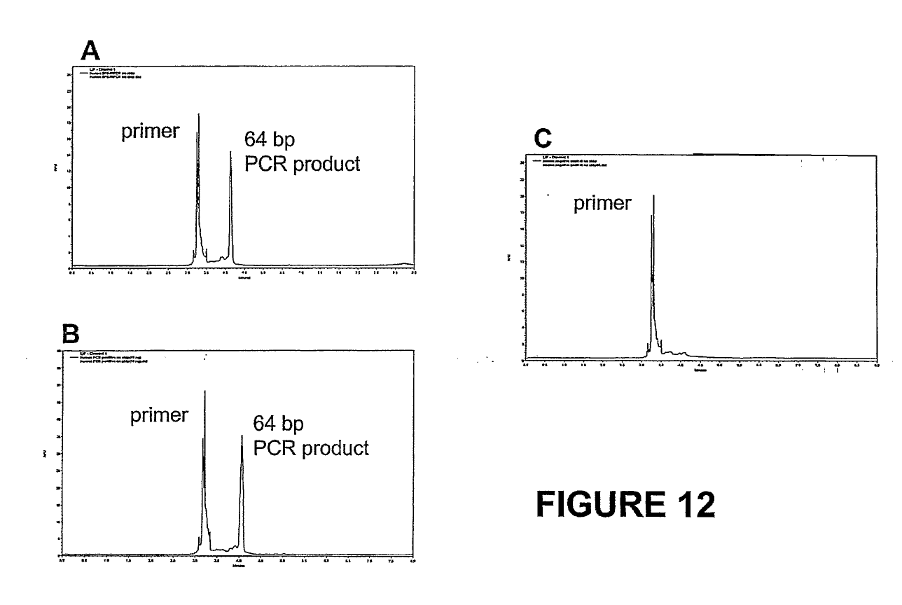

SPE-PCR was performed in the PCR chamber for human genomic SPEPCR, the result of which is shown in FIG. 12. In FIG. 12A, 10 ng of prepurified human genomic DNA was loaded onto the magnetic chitosan beads in the PCR chamber. The DNA was eluted using PCR master mix (10 mM tris 50 mM KCl pH 9, 25 mM MgCl2, 0.2 μM each primer, 0.2 mM DNTP and 0.1 U/μL Taq polymerase) and then thermocycled using the non-contact thermocycling system. Capillary electrophoresis was performed on the PCR product and the separation shows the specific 68-bp fragment expected.

In FIG. 12B, 10 ng prepurified human genomic DNA was added to the PCR master mix, same as stated above, along with 2 uL of 30 mg/mL amount of chitosan beads and PCR was performed using the non-contact thermocycling system. The presence of a 64-bp fragment indicates that the PCR was successful in the presence of the chitosan beads.

FIG. 12C shows the PCR master mix, without DNA. The beads were flowed into the chamber and non-contact PCR was performed, resulting in no specific amplification.

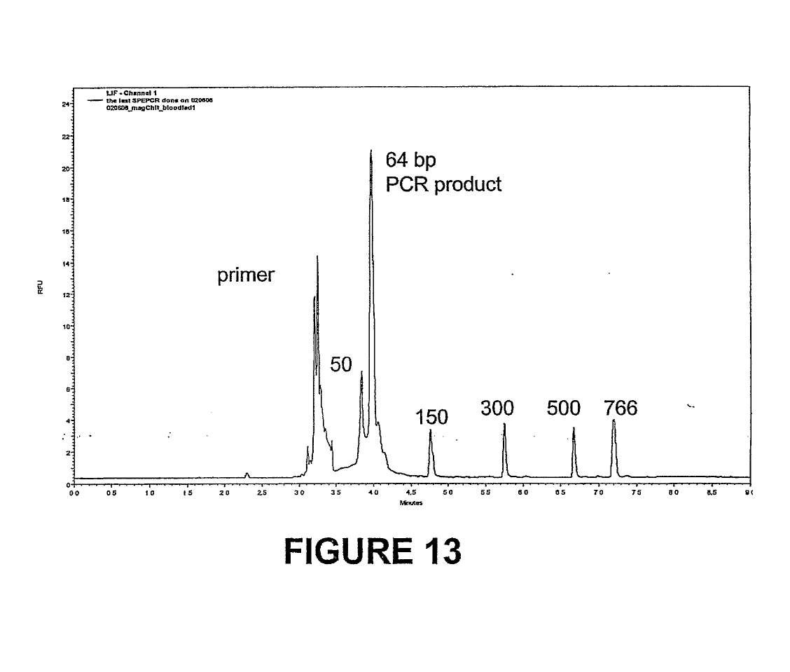

SPE PCR was performed in the PCR chamber for a blood sample, the result of which is shown in FIG. 13. 0.2 uL blood was loaded in 10 mM MES onto 1 uL of 5 mg/mL beads in the PCR chamber. The beads were washed with 10 mM MES and eluted using PCR master mix, the same as noted previously for FIG. 12. A DNA standard was coinjected with the PCR products during capillary electrophoresis to confirm the size of the PCR product from the analysis.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A method for purifying nucleic acid comprising the step of contacting a sample with a chitosan immobilized to a matrix.

2. The method of claim 1, wherein the matrix-immobilized chitosan is coated on a bead.

3. The method of claim 2, wherein the beads are silica or magnetic.

4. The method of claim 1, wherein the matrix-immobilized chitosan is contained in a micro total-analysis system.

5. The method of claim 1, wherein the chitosan and the matrix are crosslinked.

6. The method of claim 1, wherein the matrix is a sol-gel.

7. The method of claim 1, wherein the contacting step takes place at pH of about 5.

8. The method of claim 1, further comprising the step of eluting the nucleic acid from the matrix-immobilized chitosan.

9. The method of claim 8, wherein the eluting step takes place at a pH greater than about 8.5.

10. The method of claim 8, wherein the eluted nucleic acid is amplified.

11. The method of claim 8, wherein the eluted nucleic acid is amplified in the presence of the matrix-immobilized chitosan.

12. The method of claim 1, wherein the nucleic acid is DNA or RNA.

13. The method of claim 1, wherein the matrix is a polymer.

14. The method of claim 1, wherein the chitosan is covalently immobilized to the matrix.

15. The method of claim 1, wherein the chitosan is physically entrapped in the matrix.

16. The method of claim 1, further comprising the step of processing the nucleic acid.

17. The method of claim 16, wherein the processing step is selected from the group consisting of polymerase chain reaction or hybridization.

18. The method of claim 16, wherein the processing step takes place in presence of the matrix-immobilized chitosan.

19. An composition for purifying nucleic acid comprising a chitosan copolymer.

20. The composition of claim 19, wherein the chitosan copolymer is coated on beads.

21. The composition of claim 20, wherein the beads are silica or magnetic.

22. The composition of claim 19, wherein the chitosan and the matrix are crosslinked.

23. The composition of claim 19, wherein the matrix is a sol-gel.

24. The composition of claim 19, wherein the matrix is a polymer.

25. The composition of claim 19, wherein the chitosan is covalently immobilized to the matrix.

26. The method of claim 19, wherein the chitosan is physically entrapped in the matrix.

27. A microfluidic device comprising a microchamber or microchannel having a chitosan immobilized to a matrix therein.

28. The microfluidic device of claim 27, wherein the matrix and the chitosan are crosslinked.

29. The microfluidic device of claim 27, wherein the matrix is a sol-gel.

30. The microfluidic device of claim 27, wherein the matrix-immobilized chitosan is coated on a bead.

31. The microfluidic device of claim 30, wherein the beads are silica or magnetic.

32. The microfluidic device of claim 27, wherein the matrix-immobilized chitosan is immobilized on the wall of the microchamber or microchannel.

33. The microfluidic device of claim 27, wherein the matrix is a polymer.

34. The microfluidic device of claim 27, wherein the chitosan is covalently immobilized to the matrix.

35. The microfluidic device of claim 27, wherein the chitosan is physically entrapped in the matrix.
